(19) United States
(12) Patent Application Publication
(10) Pub. No.: US 2013/0338190 A1
(43) Pub. Date: Dec. 19, 2013

(54) PHARMACEUTICALLY ACCEPTABLE SALT OF (E)-N-[4-[[3-CHLORO-4-(2-PYRIDYLMETHOXY)PHENYL]AMINO]-3-CYANO-7-ETHOXY-6-QUINOLYL]-3-[(2R)-1-METHYLPYRROLIDIN-2-YL]PROP-2-ENAMIDE, PREPARATION METHOD THEREOF, AND MEDICAL USE THEREOF

(71) Applicants: Xin Li, Shanghai (CN); Bin Wang, Shanghai (CN)

(72) Inventors: Xin Li, Shanghai (CN); Bin Wang, Shanghai (CN)

(73) Assignees: JIANGSU HENGRUI MEDICINE CO., LTD., Lianyungang, Jiangsu (CN); SHANGHAI HENGRUI PHARMACEUTICAL CO., LTD., Shanghai (CN)

(21) Appl. No.: 14/001,778

(22) PCT Filed: Feb. 10, 2012
(86) PCT No.: PCT/CN2012/071015

§ 371 Date: Aug. 27, 2013

(30) Foreign Application Priority Data
Mar. 11, 2011 (CN) 201110062359.8

(51) Int. Cl. C07D 401/14; C07C 57/13; C07C 309/04; C07C 57/145; C07C 309/30

(52) U.S. Cl. CPC C07D 401/14; C07C 57/145; C07C 309/30; C07C 309/04; C07C 57/13
USPC 514/313; 546/160

(57) ABSTRACT

Provided as represented by formula (I) is a pharmaceutically acceptable salt of (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide, a preparation method thereof, and a use thereof as a therapeutic agent, and especially as a protein kinase inhibitor.

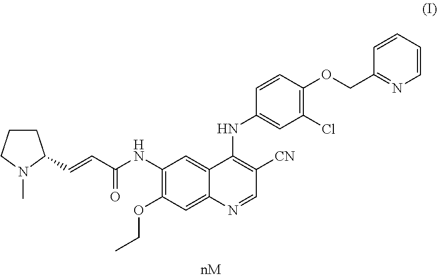 [SMILES: CCOc1cc2ncc(C#N)c(Nc3ccc(OCc4ccccn4)c(Cl)c3)c2cc1NC(=O)/C=C/[C@H]1CCCN1C]

### FIELD OF THE INVENTION

The present invention relates to a pharmaceutically acceptable salt of 6-amino quinazoline or 3-cyanoquinoline derivatives, a preparation method thereof, and a medical use thereof. Specifically, the present invention relates to a pharmaceutically acceptable salt of (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide, a preparation method thereof, and a use thereof as a therapeutic agent and especially as a protein kinase inhibitor.

### BACKGROUND OF THE INVENTION

Signal transduction is a fundamental mechanism whereby extracellular stimuli are relayed to the interior of cells, regulating corresponding physiological responses including proliferation, differentiation and apoptosis. Most of these signal transduction processes utilize the reversible phosphorylation process of proteins involving specific protein kinases and phosphatases.

There are two classes of protein kinases (PKs): the protein tyrosine kinases (PTKs) and the serine-threonine kinases (STKs). PTKs can phosphorylate tyrosine residues on a protein. STKs can phosphorylate serine and/or threonine residues. Tyrosine kinases can be divided into either the receptor-type (receptor tyrosine kinase, RTKs) or the non-receptor type (non-receptor tyrosine kinase). Now, about 90 tyrosine kinases have been identified in the human genome, of which about 60 belong to the receptor type and about 30 belong to the non-receptor type.

The Receptor Tyrosine Kinases (RTKs) family includes many subfamilies, such as (1) the epidermal growth factor (EGF) receptor family including epidermal growth factor receptor (EGFR), human epidermal growth factor 2 (HER-2), human epidermal growth factor 3 (HER-3) and human epidermal growth factor 4 (HER-4); (2) the insulin receptor family including insulin receptor (IR), insulin-like growth factor-I receptor (IGF-IR) and insulin-related receptor (IRR); (3) the Class III family such as the platelet-derived growth factor receptor (PDGFR), the stem cell factor (SCF) receptor (c-Kit) RTK, the fms-related tyrosine kinase 3 (Flt3) receptor and the colony-stimulating factor 1 receptor (CSF-1R) and the like. In addition, hepatocyte growth factor receptor c-Met, vascular endothelial growth factor (VEGFR) and the like also belong to the RTK family. They play a critical role in the control of cell growth and differentiation and are key mediators of cellular signals leading to the production of cytokines such as growth factors (Schlessinger and Ullrich, Neuron 1992, 9, 383).

The EGFR (ErbB, HER) subfamily plays a critical role in the control of cell proliferation and survival. These RTKs consist of an extracellular glycosylated ligand binding domain, a transmembrane domain and an intracellular cytoplasmic catalytic domain. The enzymatic activity of receptor tyrosine kinases can be stimulated by ligand-mediated homodimerization or heterodimerization. Dimerization results in phosphorylation of tyrosine residues on the receptors in the catalytic domain, producing a future binding site. This is followed by the activation of intracellular signaling pathways such as those involving the microtubule associated protein kinase (MAP kinase) and the phosphatidylinositol 3-kinase (PI3 kinase). Activation of these pathways is related to the regulation of cell-cycle and apoptosis. It has been identified that such mutated and over expressed forms of tyrosine kinases, like EGFR, HER-2, are present in a large proportion of common human cancers such as breast cancer, prostate cancer, non-small cell lung cancer, esophageal cancer, ovarian cancer and pancreatic cancer and the like. The prevalence and relevance of tyrosine kinases is confirmed in oncogenesis and cancer growth.

It is expected to synthesize novel compounds having anti-tumor cell proliferative activities. These compounds are expected to inhibit one or more RTKs or STKs, and are useful for treating or ameliorating RTK- or STK-mediated, and angiogenesis mediated physiological disorders with cell over-proliferation.

The present invention disclosure relates to a series of pharmaceutically acceptable salts of (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide and their uses, especially as protein kinase inhibitors.

The inventor finds that the free base of (E)-N-[4-[[3-chloro-4-(2-pyridylmethoxy) phenyl]amino]-3-cyano-7-ethoxy-6-quinolyl]-3-[(2R)-1-methylpyrrolidin-2-yl]prop-2-enamide is poorly soluble in conventional solvents and is thus disadvantageous to be prepared into a medicinal dosage form, limiting their in vivo bioavailability. Thus, there is an urgent need to improve its solubility and pharmacokinetic absorption in order to be suitable for the conventional preparation process of dosage forms. Compared with the free base of (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide, the solubility and pharmacokinetics of the pharmaceutically acceptable salts of the compound are significantly improved, and the synthetic process is simplified.

The present invention is directed to providing pharmaceutically acceptable salts of the compound of formula (I), thereby improving their physical/chemical properties and pharmacokinetic characteristics.

### SUMMARY OF THE INVENTION

The present invention relates to providing a pharmaceutically acceptable salt of formula (I), and a preparation method thereof. Preferably, the dimaleate salt of formula (I) has advantages in solubility, bioavailability and pharmacokinetics compared with the compound of formula (I) itself and other salts thereof:

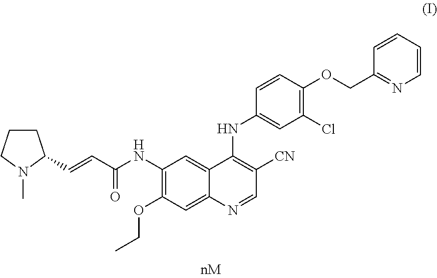

wherein:
n is 1, 2 or 3; and
M is an acid molecule.

In a first aspect, the present invention relates to a pharmaceutically acceptable salt of (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide of formula (I), wherein said salt is a conventional inorganic salt or organic salt in the art. Further, said inorganic salt is selected from the group consisting of hydrochloride, hydrobromide, sulfate, nitrate and phosphate, preferably hydrochloride, more preferably dihydrochloride; said organic salt is selected from the group consisting of p-toluenesulfonate, methanesulfonate, maleate, tartrate, succinate, acetate, trifluoroacetate, fumarate, citrate, benzene sulfonate, benzoate, naphthalene sulfonate, lactate and L-malate, preferably L-malic acid, methanesulfonate, succinate, p-toluenesulphonate or maleate, most preferably maleate. Especially the maleate salt of the compound of formula (I) has advantages in solubility, bioavailability and pharmacokinetics compared with the compound of formula (I) itself and other salts thereof.

In a second aspect, the present invention relates to a process of preparing the pharmaceutically acceptable salt of (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide, and this compound can be prepared according to the conventional salifying process in the art. Specifically, said process comprises the step of reacting (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide with a corresponding acid to form the salt, wherein said acid is an inorganic acid or organic acid selected from the group consisting of phosphoric acid, hydrochloric acid, sulfuric acid, nitric acid, hydrobromic acid, p-toluenesulfonic acid, methanesulfonic acid, maleic acid, tartaric acid, succinic acid, acetic acid, trifluoroacetic acid, fumaric acid, citric acid, benzenesulfonic acid, benzoic acid, naphthalene sulfonic acid, lactic acid and L-malic acid.

Pharmaceutically acceptable salts of the present invention typically include, but are not limited to:

| Example | |
|---|---|
| No. | Structure and Name |
| 1 | 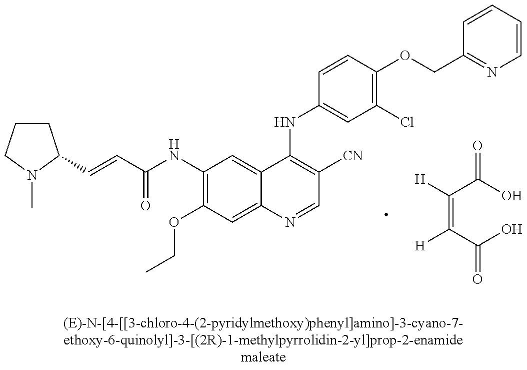 [SMILES: CCOc1cc2ncc(C#N)c(Nc3ccc(OCc4ccccn4)c(Cl)c3)c2cc1NC(=O)/C=C/[C@H]1CCCN1C ; O=C(O)/C=C\C(=O)O] |
| 2 | 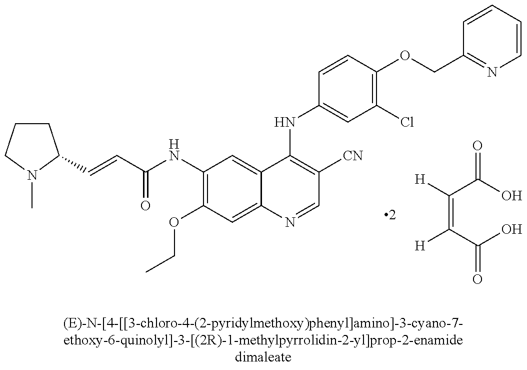 [SMILES: CCOc1cc2ncc(C#N)c(Nc3ccc(OCc4ccccn4)c(Cl)c3)c2cc1NC(=O)/C=C/[C@H]1CCCN1C ; O=C(O)/C=C\C(=O)O] |
| 3 | 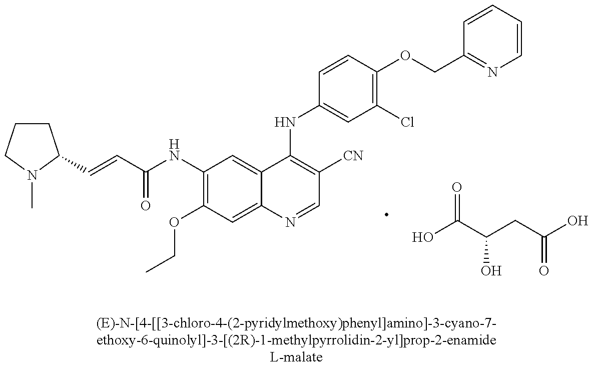 [SMILES: CCOc1cc2ncc(C#N)c(Nc3ccc(OCc4ccccn4)c(Cl)c3)c2cc1NC(=O)/C=C/[C@H]1CCCN1C ; O=C(O)C[C@H](O)C(=O)O] |
| 4 | 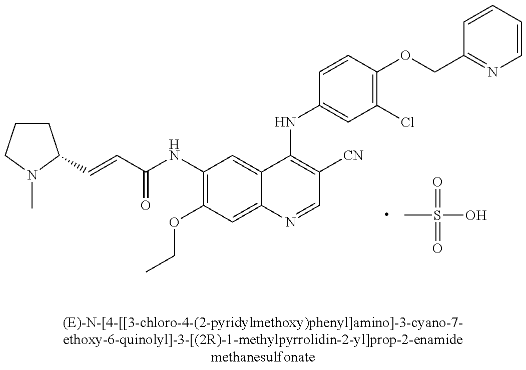 |
| 5 | 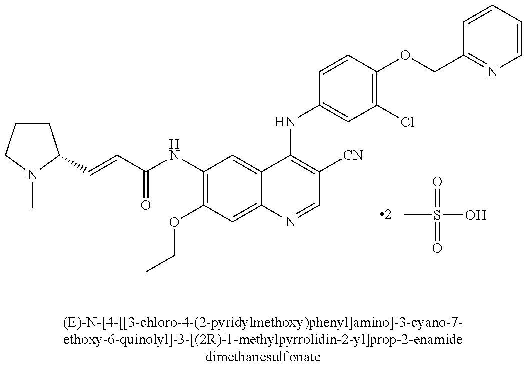 |
| 6 | 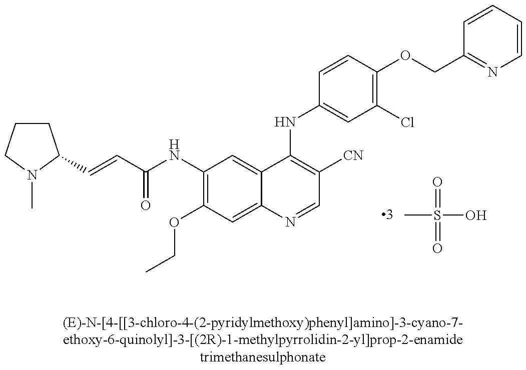 |
| 7 | 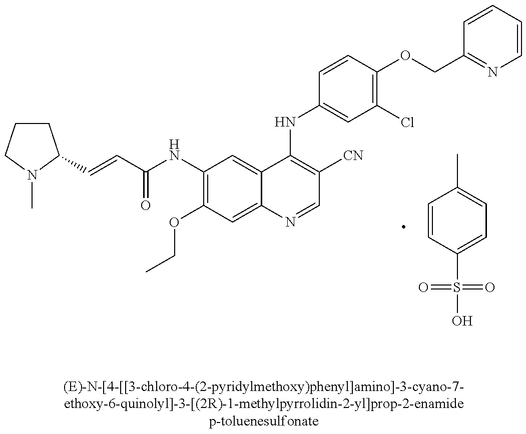 [SMILES: CCOc1cc2ncc(C#N)c(Nc3ccc(OCc4ccccn4)c(Cl)c3)c2cc1NC(=O)/C=C/[C@H]1CCCN1C ; Cc1ccc(S(=O)(=O)O)cc1] |
| 8 | 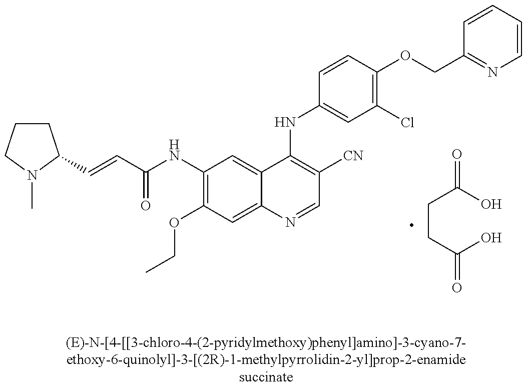 [SMILES: CCOc1cc2ncc(C#N)c(Nc3ccc(OCc4ccccn4)c(Cl)c3)c2cc1NC(=O)/C=C/[C@H]1CCCN1C ; O=C(O)CCC(=O)O] |
| 9 | 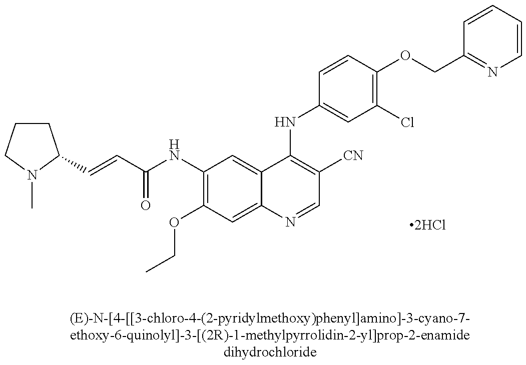 [SMILES: CCOc1cc2ncc(C#N)c(Nc3ccc(OCc4ccccn4)c(Cl)c3)c2cc1NC(=O)/C=C/[C@H]1CCCN1C] |

In a third aspect, the present invention relates to a pharmaceutical composition comprising a therapeutically effective amount of a pharmaceutically acceptable salt of (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide and pharmaceutically acceptable carriers. The present invention also relates to a process for preparing the composition comprising a step of combining the pharmaceutically acceptable salt with a pharmaceutically acceptable carrier or diluent.

In a fourth aspect, the present invention relates to a use of a pharmaceutically acceptable salt of (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide or a pharmaceutical composition thereof in the preparation of a medicament for the treatment of protein kinase related diseases, wherein said protein kinase is selected from the group consisting of EGFR receptor tyrosine kinases and HER-2 receptor tyrosine kinases.

In a fifth aspect, the present invention relates to a method for the treatment of protein kinase related diseases, comprising administrating a therapeutically effective amount of a pharmaceutically acceptable salt of (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide or a pharmaceutical composition thereof to a subject in need of it.

In a sixth aspect, the present invention relates to a use of a pharmaceutically acceptable salt of (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide or a pharmaceutical composition thereof in the preparation of a medicament of a protein kinase inhibitor, wherein said protein kinase is selected from the group consisting of EGFR and HER-2.

In a seventh aspect, the present invention relates to a use of a pharmaceutically acceptable salt of (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide or a pharmaceutical composition thereof as a medicament for the treatment of protein kinase related diseases, wherein said diseases are cancers selected from the group consisting of lung cancer, breast cancer, squamous cell carcinoma and stomach cancer.

In an eighth aspect, the present invention relates to a method for regulating the catalytic activity of protein kinases comprising contacting said protein kinase with the pharmaceutically acceptable salt of (E)-N-[4-[[3-chloro-4-(2-pyridylmethoxy) phenyl]amino]-3-cyano-7-ethoxy-6-quinolyl]-3-[(2R)-1-methylpyrrolidin-2-yl]prop-2-enamide, wherein said protein kinase is selected from the group consisting of EGFR receptor tyrosine kinase or HER-2 receptor tyrosine kinase.

### DETAILED DESCRIPTION OF THE INVENTION

The present invention is further described by the following Examples, which are not intended to limit the scope of the invention.

### EXAMPLES

The structures of all compounds were identified by nuclear magnetic resonance (NMR) and/or mass spectrometry (MS). NMR chemical shifts (δ) were recorded as ppm (10⁻⁶).

NMR was performed on a Bruker AVANCE-400 spectrometer. The detection solvent was deuterated-dimethyl sulfoxide (d-DMSO) with tetramethylsilane (TMS) as the internal standard, and the chemical shift was recorded as ppm (10⁻⁶).

MS was determined on a FINNIGAN LCQAd (ESI) mass spectrometer (Thermo, Model: Finnigan LCQ advantage MAX).

High performance liquid chromatography (HPLC) was performed on an Agilent 1200DAD high pressure liquid chromatography spectrometer (Sunfire C18 150×4.6 mm chromatographic column) and a Waters 2695-2996 high pressure liquid chromatography spectrometer (Gimini C18 150×4.6 mm chromatographic column).

Column chromatography generally used Yantai Huanghai 200-300 mesh silica gel as carrier.

The starting materials of the present invention are known and can be purchased from ABCR GmbH & Co. KG, Acros Organics, Aldrich Chemical Company and so on, or they can be prepared by the conventional synthesis methods known in the art.

Unless otherwise stated, the following reactions were placed under argon atmosphere or nitrogen atmosphere.

The term “argon atmosphere” or “nitrogen atmosphere” means that a reaction flask was equipped with a balloon filled with about 1 L of argon or nitrogen.

The term “hydrogen atmosphere” means that a reaction flask was equipped with a balloon filled with about 1 L of hydrogen.

Unless otherwise stated, the solution used in the Examples refers to an aqueous solution.

Unless otherwise stated, the reaction temperature was room temperature (r.t.).

Room temperature is the most proper reaction temperature, which is 20° C.-30° C.

The reaction processes in the Examples were monitored by thin layer chromatography (TLC). The developing solvent systems comprised a dichloromethane and methanol system, n-hexane and ethyl acetate system, petroleum ether and ethyl acetate system, and acetone. The ratio by volume of the solvent was adjusted according to the polarity of the compounds.

The elution systems of column chromatography comprised A: dichloromethane, methanol and acetone system; B: hexane and ethyl acetate system. The ratio by volume of the solvent was adjusted according to the polarity of the compounds, and sometimes a little ammonia and acetic acid could also be added.

### Example 1

### (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide maleate

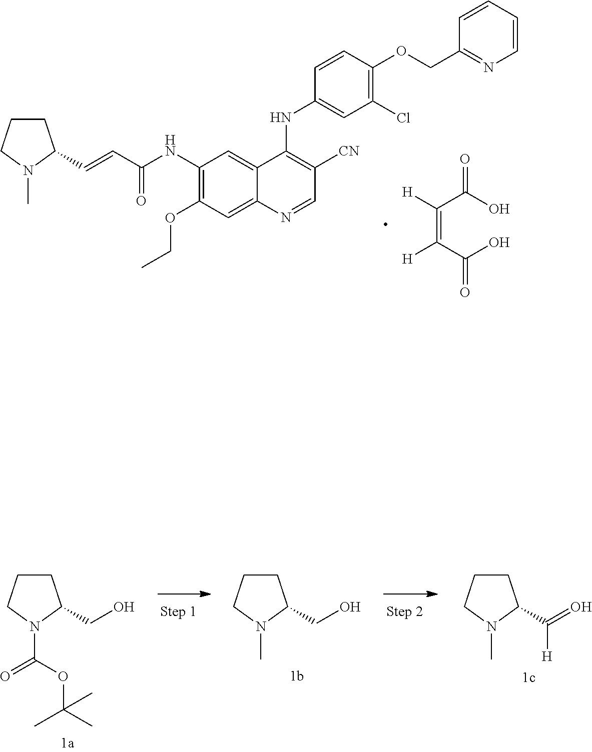

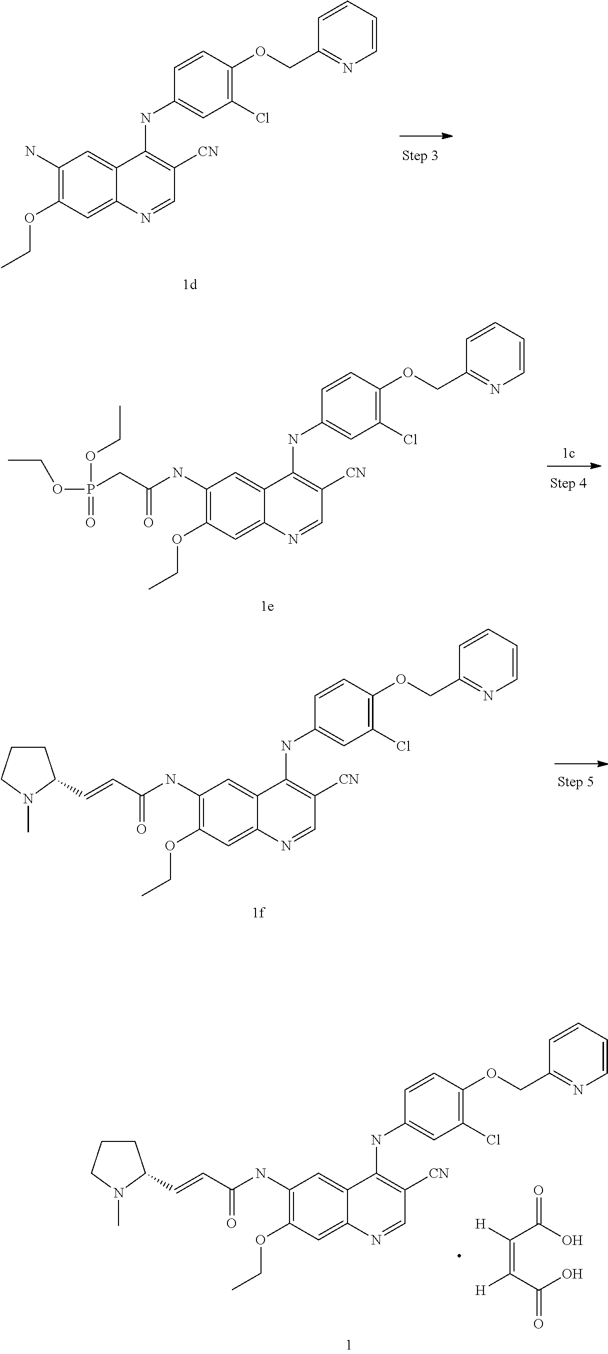 [SMILES: CCOc1cc2ncc(C#N)c(Nc3ccc(OCc4ccccn4)c(Cl)c3)c2cc1N ; CCOc1cc2ncc(C#N)c(Nc3ccc(OCc4ccccn4)c(Cl)c3)c2cc1NC(=O)CP(=O)(OCC)OCC ; CCOc1cc2ncc(C#N)c(Nc3ccc(OCc4ccccn4)c(Cl)c3)c2cc1NC(=O)/C=C/[C@H]1CCCN1C ; CCOc1cc2ncc(C#N)c(Nc3ccc(OCc4ccccn4)c(Cl)c3)c2cc1NC(=O)/C=C/[C@H]1CCCN1C ; O=C(O)/C=C\C(=O)O]

### Step 1

### [(2R)-1-Methylpyrrolidin-2-yl]methanol

Lithium aluminium hydride (230 mg, 6 mmol) and N-tert-butoxycarbonyl-R-prolinol 1a (400 mg, 2 mmol) were dissolved in 10 mL of dry tetrahydrofuran in an ice-water bath in batches. After no gas was obviously released, the reaction mixture was heated to reflux for 2 hours. The reaction mixture was added dropwise to 5 mL of methanol in an ice-water bath, followed by addition of 5 mL of water, dried over anhydrous magnesium sulfate, filtered and concentrated under reduced pressure to give the title compound [(2R)-1-methylpyrrolidin-2-yl]methanol 1b (221 mg, yield 77.0%) as a colourless oil. MS m/z (ESI): 116 [M+1].

### Step 2

### (2R)-1-Methylpyrrolidine-2-formaldehyde

Dimethyl sulfoxide (820 μL, 11.46 mmol) was dissolved in 5 mL of dichloromethane in a dry ice bath, followed by the dropwise slow addition of oxalyl chloride (968 mg, 7.64 mmol). After stirring for 45 minutes, a solution of [(2R)-1-methylpyrrolidin-2-yl]methanol 1b (220 mg, 1.91 mmol) in 2 mL of dichloromethane was added to the solution. The reaction mixture was stirred for another 45 minutes, and triethylamine (1.9 mL, 13.37 mmol) was added. The reaction mixture was stirred for 10 minutes, then warmed up to room temperature and stirred for 1 hour. The reaction mixture was washed with water (20 mL) and saturated brine (10 mL) successively. The combined organic extracts were dried over anhydrous sodium sulfate, filtered and concentrated under reduced pressure, and the resulting residues were purified by alkaline alumina column chromatography with elution system A to give the title compound (2R)-1-methylpyrrolidine-2-formaldehyde 1c (300 mg) as a yellow solid, which was directly used in the next step without purification.

### Step 3

### N-[4-[[3-Chloro-4-(2-pyridylmethoxy)phenyl]amino]-3-cyano-7-ethoxy-6-quinolyl]-2-diethoxyphosphoryl-acetamide

N,N′-Carbonyldiimidazole (487 mg, 3 mmol) was dissolved in 4 mL of tetrahydrofuran. The mixture was heated to 40° C. in an oil bath, a solution of diethylphosphonoacetic acid (588 mg, 3 mmol) in tetrahydrofuran (4 mL) was added dropwise to the mixture, and stirred for 30 minutes prior to the next step.

6-Amino-4-[[3-chloro-4-(2-pyridylmethoxy)phenyl]amino]-7-ethoxy-quinoline-3-carbonitrile 1d (446 mg, 1 mmol, prepared by the well-known method in PCT Patent Application Publication No. WO2005028443) was dissolved in 4 mL of tetrahydrofuran at 40° C., followed by dropwise addition of the above reaction solution. After stirring for 12 hours, the reaction mixture was concentrated under reduced pressure and extracted with dichloromethane (50 mL×3). The combined organic extracts were washed with saturated brine (30 mL×2), dried over anhydrous sodium sulfate, filtered and concentrated under reduced pressure, and the resulting residues were purified by silica gel column chromatography with elution system A to give the title compound N-[4-[[3-chloro-4-(2-pyridylmethoxy)phenyl]amino]-3-cyano-7-ethoxy-6-quinolyl]-2-diethoxyphosphoryl-acetamide 1e (624 mg, yield 99.9%) as a light yellow solid. MS m/z (ESI): 624 [M+1].

### Step 4

### (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide

N-[4-[[3-Chloro-4-(2-pyridylmethoxy)phenyl]amino]-3-cyano-7-ethoxy-6-quinolyl]-2-diethoxyphosphoryl-acetamide 1e (250 mg, 0.40 mmol) was dissolved in 10 mL of dry tetrahydrofuran in a dry ice bath, followed by dropwise addition of a solution of lithium bis(trimethylsilyl)amide (1 M) in toluene (440 μL, 0.44 mmol). The reaction mixture was stirred for 30 minutes, added dropwise to a solution of (2R)-1-methylpyrrolidine-2-formaldehyde 1c (90 mg, 0.80 mmol) in tetrahydrofuran (5 mL), and stirred for 30 minutes, then warmed up to room temperature and stirred for 12 hours. The reaction mixture was concentrated under reduced pressure and the resulting residue was purified by silica gel column chromatography with elution system A to give the title compound (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide 1f (46 mg, yield 19.7%) as a yellow solid. MS m/z (ESI): 583.4 [M+1]; ¹H NMR (400 MHz, DMSO-d₆): δ 9.16 (s, 1H), 8.63 (d, 1H), 8.56 (s, 1H), 8.26 (s, 1H), 7.83-7.80 (dd, 1H), 7.76-7.50 (m, 2H), 7.57-7.56 (m, 1H), 7.40 (s, 1H), 7.38 (s, 1H), 7.19 (d, 1H), 7.06-7.03 (m, 2H), 6.34-6.31 (d, 1H), 5.35 (s, 2H), 4.39 (m, 2H), 4.27-4.26 (m, 1H), 3.32 (m, 1H), 3.10 (m, 1H), 2.73 (s, 3H), 2.37-2.36 (m, 2H), 2.07-2.01 (m, 2H), 1.64 (t, 3H).

### Step 5

### (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide maleate

(E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide 1f (500 mgl 0.86 mmol) and maleic acid (109 mg, 0.94 mmol) were dissolved in 5 mL of dichloromethane with stirring. After stirring for 1 hour under r.t., the reaction mixture was concentrated under reduced pressure to give (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide maleate 1 (609 mg, yield 100%) as a yellow solid. MS m/z (ESI): 584.4 [M+1-116]; ¹H NMR (400 MHz, CDCl₃): δ 9.22 (s, 1H), 8.63 (m, 1H), 8.52 (m, 1H), 8.42 (s, 1H), 7.84 (m, 1H), 7.74 (m, 1H), 7.69 (m, 1H), 7.58 (m, 1H), 7.26 (m, 1H), 7.02 (m, 2H), 6.92 (m, 1H), 6.72 (m, 1H), 6.25 (s, 2H), 5.27 (s, 2H), 4.27 (m, 2H), 3.90 (m, 2H), 3.00 (m, 1H), 2.87 (m, 2H), 2.21 (m, 4H), 2.09 (m, 1H), 1.56 (t, 3H, J=8 Hz).

### Example 2

### (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide dimaleate

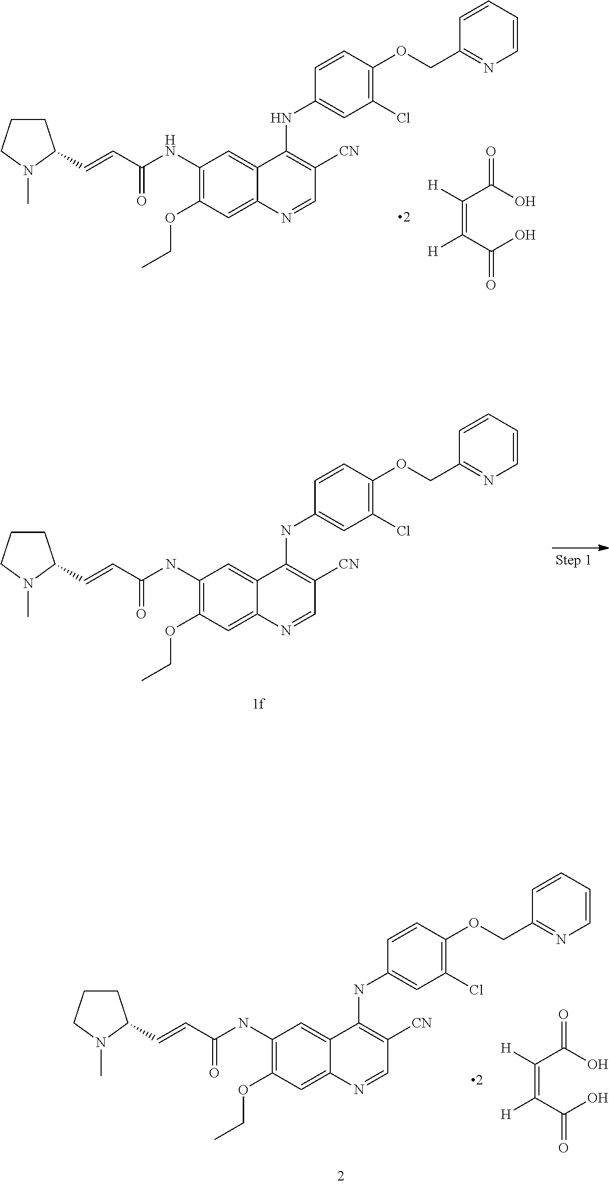 [SMILES: CCOc1cc2ncc(C#N)c(Nc3ccc(OCc4ccccn4)c(Cl)c3)c2cc1NC(=O)/C=C/[C@H]1CCCN1C ; O=C(O)/C=C\C(=O)O ; CCOc1cc2ncc(C#N)c(Nc3ccc(OCc4ccccn4)c(Cl)c3)c2cc1NC(=O)/C=C/[C@H]1CCCN1C ; CCOc1cc2ncc(C#N)c(Nc3ccc(OCc4ccccn4)c(Cl)c3)c2cc1NC(=O)/C=C/[C@H]1CCCN1C ; O=C(O)/C=C\C(=O)O]

### Step 1

### (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide dimaleate

(E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide 1f (200 mg, 0.34 mmol) was dissolved in 6 mL of ethanol with stirring, followed by addition of maleic acid (80 mg, 0.68 mmol). After stirring for 0.5 hour and standing for 12 hours under r.t., the reaction mixture was mixed with 10 mL of diethyl ether and filtered. The solid was washed with diethyl ether (20 mL) and dried in vacuo to give the title product (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide dimaleate 2 (220 mg, yield 78.6%) as a yellow solid. MS m/z (ESI): 583.4 [M+1-232]; ¹H NMR (400 MHz, CDCl₃): δ 9.12 (s, 1H), 8.57 (m, 1H), 8.51 (m, 1H), 8.37 (s, 1H), 7.79 (m, 1H), 7.71 (m, 1H), 7.63 (m, 1H), 7.52 (m, 1H), 7.21 (m, 1H), 7.00 (m, 2H), 6.90 (m, 1H), 6.57 (m, 1H), 6.25 (s, 4H), 5.23 (s, 2H), 4.21 (m, 2H), 3.91 (m, 2H), 3.11 (m, 1H), 2.85 (m, 2H), 2.22 (m, 4H), 2.01 (m, 1H), 1.54 (t, 3H, J=8 Hz).

### Example 3

### (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide L-malate

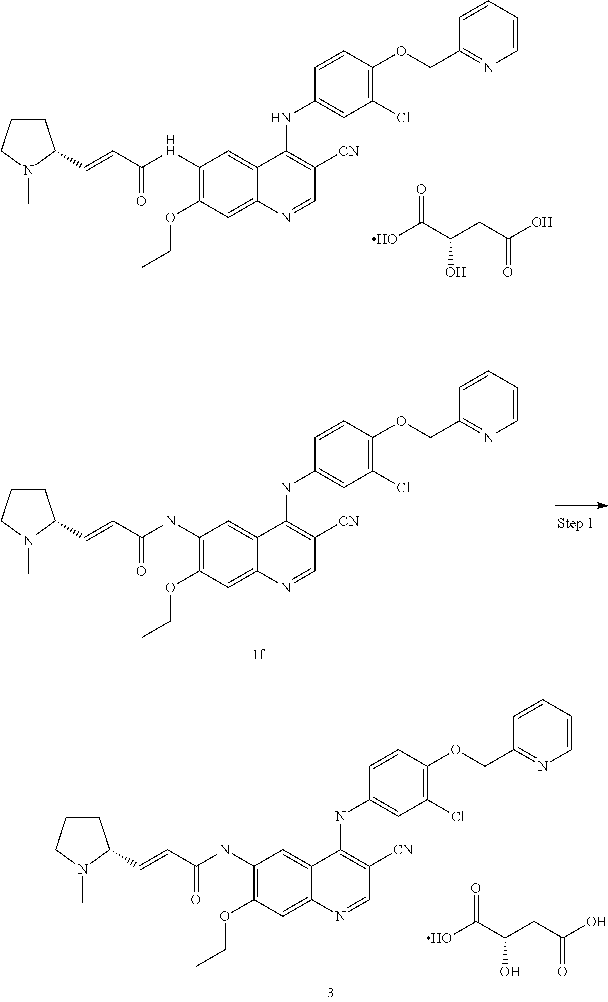

### Step 1

### (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide L-malate

(E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide 1f (300 mg, 0.51 mmol) was dissolved in 5 mL of dichloromethane with stirring, followed by addition of L-malic acid (75.9 mg, 0.56 mmol). After stirring for 4 hours under r.t., the reaction mixture was concentrated under reduced pressure and dried in vacuo to give (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide L-malate 3 (375 mg, yield 100%) as a yellow solid. MS m/z (ESI): 583.4 [M+1-134]; ¹H NMR (400 MHz, MeOD): δ 8.96 (s, 1H), 8.58 (m, 1H), 8.38 (s, 1H), 7.94 (m, 1H), 7.73 (m, 1H), 7.42 (m, 2H), 7.24 (s, 1H), 7.19 (m, 2H), 6.98 (m, 1H), 6.71 (m, 1H), 5.51 (s, 1H), 5.28 (m, 2H), 4.34 (m, 2H), 3.64 (m, 2H), 2.92 (m, 2H), 2.67 (m, 3H), 2.58 (m, 1H), 2.31 (m, 1H), 2.17 (m, 1H), 2.09 (m, 2H), 1.98 (m, 1H), 1.58 (t, 3H, J=8 Hz).

### Example 4

### (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide methanesulfonate

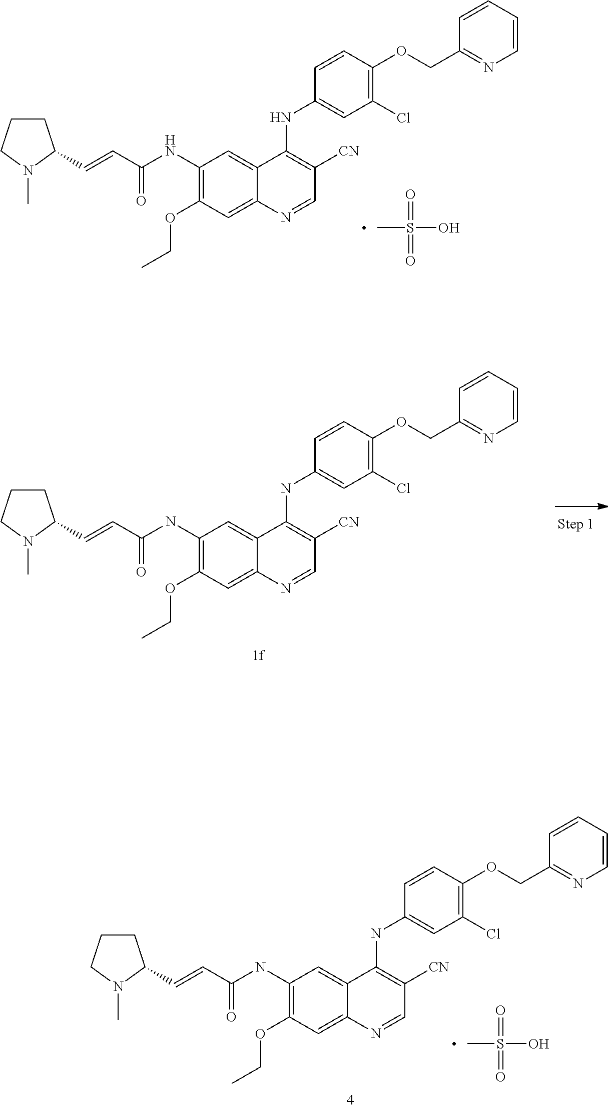

### Step 1

### (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide methanesulfonate

(E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide 1f (150 mg, 0.26 mmol) was dissolved in 2 mL of ethanol with stirring, followed by addition of methanesulfonic acid (1.67 mL, 0.26 mmol). After stirring for 12 hours under r.t., the reaction mixture was mixed with 2 mL of diethyl ether and filtered. The solid was washed with diethyl ether (10 mL) and dried in vacuo to give the title product (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide methanesulfonate 4 (120 mg, yield 69.0%) as a yellow solid. MS m/z (ESI): 583.4 [M+1-96]; ¹H NMR (400 MHz, DMSO-d₆): δ 11.10 (m, 1H), 10.00 (s, 1H), 9.91 (m, 1H), 9.11 (s, 1H), 9.01 (s, 1H), 8.60 (d, 1H, J=4 Hz), 8.03 (m, 1H), 7.57 (m, 2H), 7.47 (m, 4H), 6.92 (m, 2H), 5.31 (s, 2H), 4.43 (m, 2H), 4.01 (m, 2H), 3.73 (m, 3H), 3.14 (s, 2H), 2.80 (m, 2H), 2.32 (m, 1H), 2.27 (m, 3H), 1.51 (t, 3H, J=8 Hz).

### Example 5

### (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide dimethanesulfonate

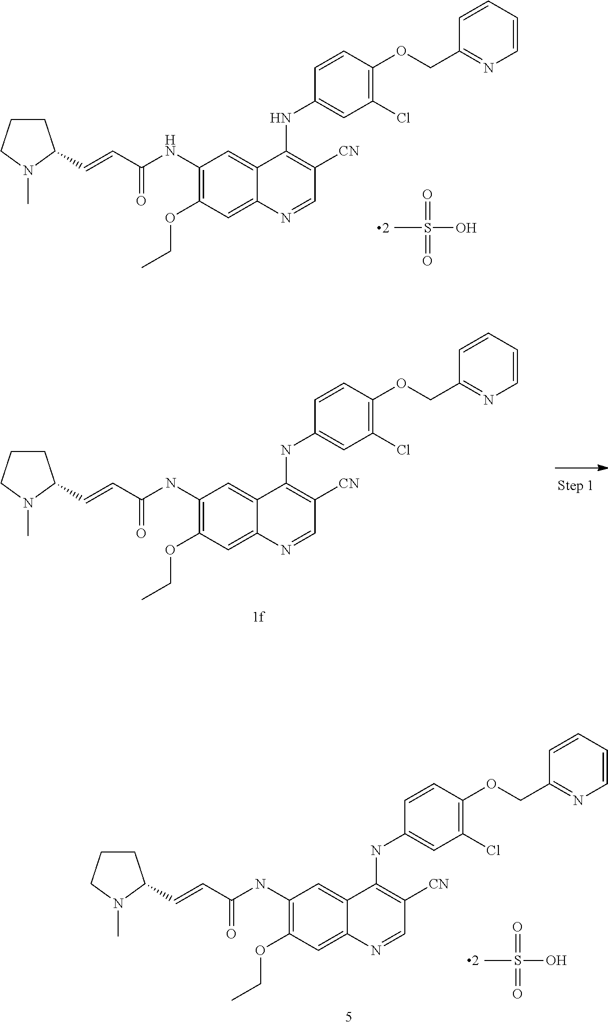

### Step 1

### (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide dimethanesulfonate

(E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide 1f (200 mg, 0.34 mmol) was dissolved in 2 mL of ethanol with stirring, followed by addition of methanesulfonic acid (4.45 mL, 0.68 mmol). After stirring for 12 hours under r.t., the reaction mixture was mixed with 2 mL of diethyl ether and filtered, and the solid was washed with diethyl ether (10 mL) and dried in vacuo to give the title product (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide dimethanesulfonate 5 (180 mg, yield 67.7%) as a yellow solid. MS m/z (ESI): 583.4 [M+1-192]; ¹H NMR (400 MHz, DMSO-d₆): δ 11.17 (m, 1H), 10.04 (s, 1H), 9.98 (m, 1H), 9.18 (s, 1H), 9.05 (s, 1H), 8.67 (d, 1H, J=4 Hz), 8.01 (m, 1H), 7.68 (m, 2H), 7.50 (m, 4H), 6.91 (m, 2H), 5.39 (s, 2H), 4.40 (m, 2H), 4.14 (m, 2H), 3.71 (m, 6H), 3.22 (s, 2H), 2.82 (m, 2H), 2.36 (m, 1H), 2.32 (m, 3H), 1.52 (t, 3H, J=8 Hz).

### Example 6

### (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide trimethanesulphonate

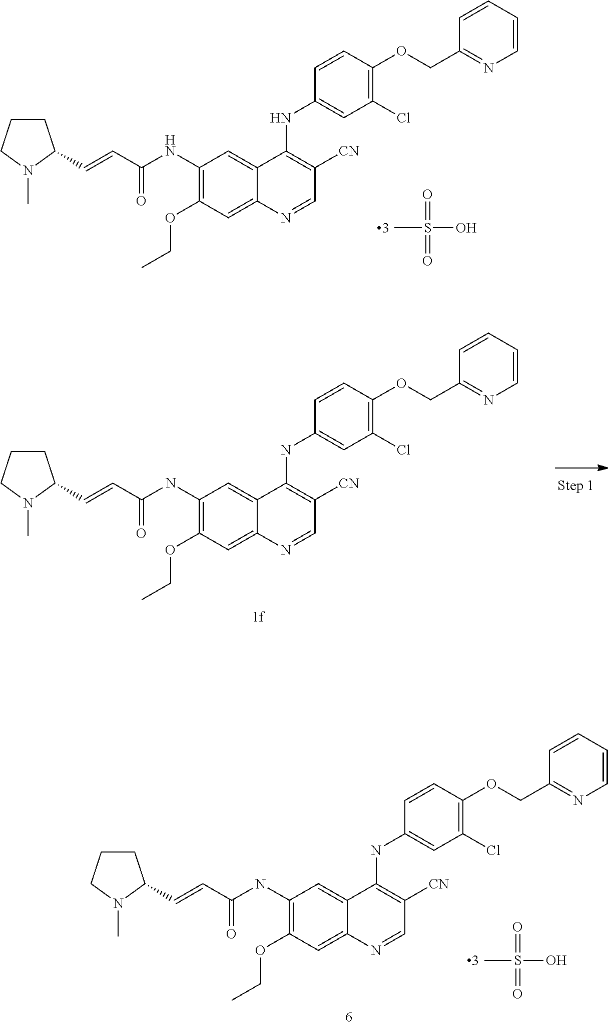

### Step 1

### (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide trimethanesulphonate

(E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide 1f (150 mg, 0.26 mmol) was dissolved in 1 mL of ethanol with stirring, followed by addition of methanesulfonic acid (5.00 mL, 0.78 mmol). After stirring for 12 hours, the reaction mixture was mixed with 2 mL of diethyl ether and filtered, the solid was washed with diethyl ether (10 mL) and dried in vacuo to give the title product (E)-N-[4-[[3-chloro-4-(2-pyridylmethoxy)phenyl]amino]-3-cyano-7-ethoxy-6-quinolyl]-[(2R)-1-methylpyrrolidin-2-yl]prop-2-enamide trimethanesulphonate 6 (140 mg, yield 62.5%) as a yellow solid. MS m/z (ESI): 583.4 [M+1-288]; ¹H NMR (400 MHz, DMSO-d₆): δ 10.97 (m, 1H), 9.99 (s, 1H), 9.89 (m, 1H), 9.27 (s, 1H), 9.08 (s, 1H), 8.71 (d, 1H, J=4 Hz), 7.93 (m, 1H), 7.62 (m, 2H), 7.51 (m, 4H), 6.94 (m, 2H), 5.27 (s, 2H), 4.37 (m, 2H), 4.09 (m, 2H), 3.67 (m, 9H), 3.19 (s, 2H), 2.78 (m, 2H), 2.31 (m, 1H), 2.27 (m, 3H), 1.52 (t, 3H, J=8 Hz).

### Example 7

### (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide p-toluenesulfonate

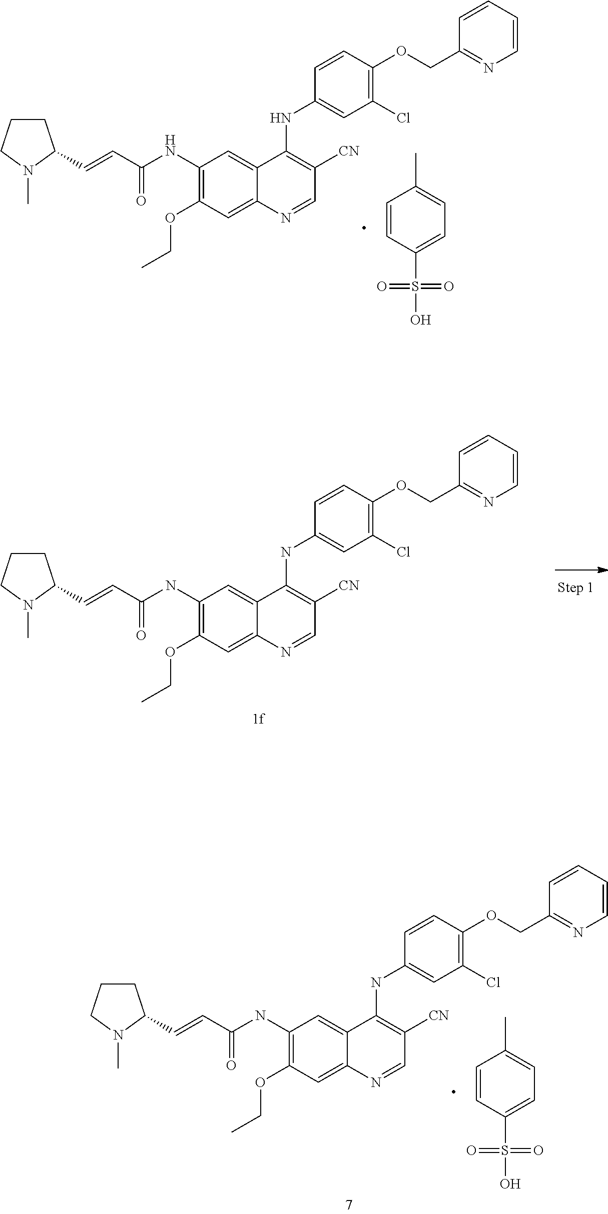

### Step 1

### (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide p-toluenesulfonate

(E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide 1f (300 mg, 0.514 mmol) was dissolved in 3 mL of n-propanol/water (3/1) mixed solvent with stirring, followed by addition of p-toluenesulfonic acid monohydrate (117 mg, 0.0.617 mmol). After stirring for 0.5 hour under r.t., the reaction mixture was concentrated under reduced pressure and dried in vacuo to give the title product (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide p-toluenesulfonate 7 (410 mg, yield 98.0%) as a yellow solid. MS m/z (ESI): 584.2 [M+1-172]: ¹H NMR (400 MHz, MeOD): δ 8.56 (s, 1H), 8.46 (s, 1H), 8.26 (s, 1H), 7.85 (m, 1H), 7.65 (m, 4H), 7.40 (m, 5H), 7.12 (m, 1H), 7.00 (m, 2H), 6.81 (m, 2H), 5.12 (s, 2H), 4.14 (m, 4H), 3.54 (m, 7H), 2.83 (m, 3H), 2.33 (m, 6H), 1.56 (t, 3H, J=7.2 Hz).

### Example 8

### (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide succinate

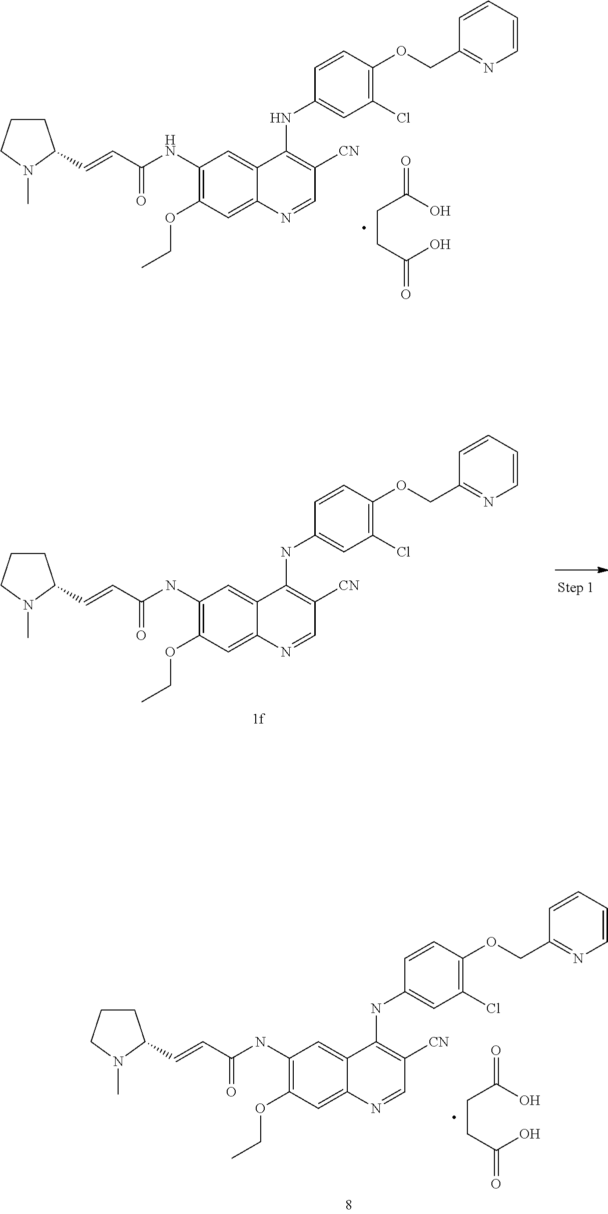

### Step 1

### (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide succinate

(E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide 1f (400 mg, 0.69 mmol) was dissolved in 5 mL of dichloromethane with stirring, followed by addition of succinic acid (89.0 mg, 0.75 mmol). After stirring for 0.5 hour under r.t., the reaction mixture was concentrated under reduced pressure and dried in vacuo to give the title product (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide succinate 8 (442 mg, yield 90.4%) as a yellow solid. MS m/z (ESI): 583.4 [M+1-118]; ¹H NMR (400 MHz, MeOD): δ 12.21 (s, 2H), 9.62 (s, 1H), 8.96 (s, 1H), 8.61 (s, 1H), 8.47 (m, 1H), 7.87 (m, 1H), 7.59 (m, 1H), 7.39 (m, 2H), 7.25 (m, 2H), 6.66 (m, 1H), 5.76 (m, 1H), 5.29 (s, 2H), 4.31 (m, 2H), 4.03 (m, 1H), 3.32 (m, 2H), 2.50 (m, 2H), 2.24 (m, 1H), 1.48 (t, 3H, J=7.2 Hz).

### Example 9

### (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide dihydrochloride

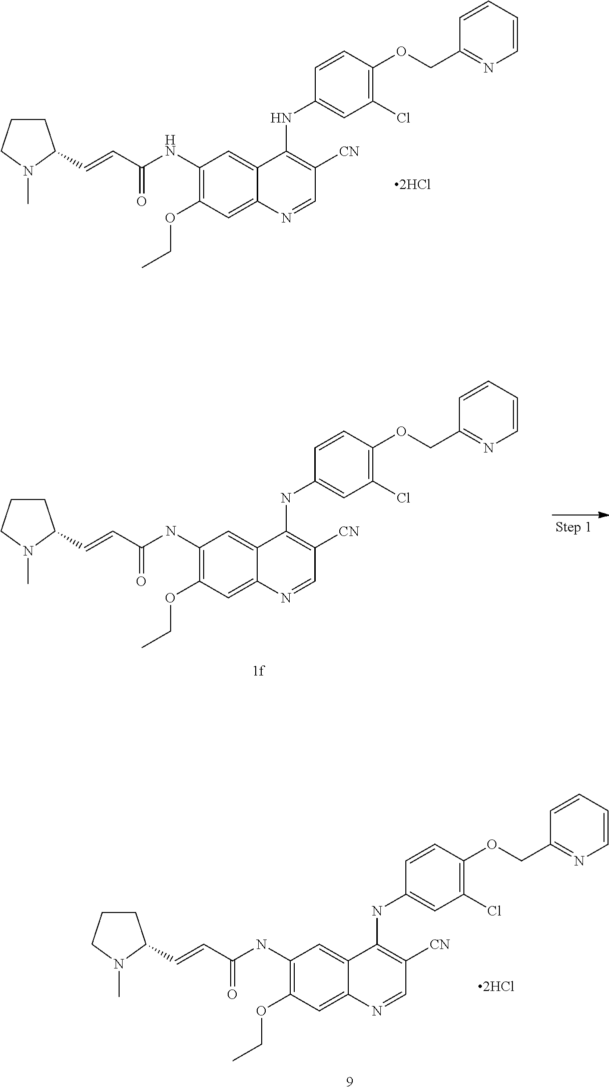 [SMILES: CCOc1cc2ncc(C#N)c(Nc3ccc(OCc4ccccn4)c(Cl)c3)c2cc1NC(=O)/C=C/[C@H]1CCCN1C ; CCOc1cc2ncc(C#N)c(Nc3ccc(OCc4ccccn4)c(Cl)c3)c2cc1NC(=O)/C=C/[C@H]1CCCN1C ; CCOc1cc2ncc(C#N)c(Nc3ccc(OCc4ccccn4)c(Cl)c3)c2cc1NC(=O)/C=C/[C@H]1CCCN1C]

### Step 1

### (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide dihydrochloride

(E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide 1f (1000 mg, 1.71 mmol) was dissolved in 5 mL of dichloromethane with stirring, followed by addition of a solution of hydrochloric acid in diethyl ether (1M, 3.42 mL, 3.42 mmol). After stirring for 0.5 hour in an ice-bath, the reaction mixture was concentrated under reduced pressure and dried in vacuo to give the title product (E)-N-[4-[[3-chloro-4-(2-pyridylmethoxy) phenyl]amino]-3-cyano-7-ethoxy-6-quinolyl]-3-[(2R)-1-methylpyrrolidin-2-yl]prop-2-enamide dihydrochloride 9 (1500 mg, yield 100%) as a yellow solid.

MS m/z (ESI): 583.2 [M+1-72]; ¹H NMR (400 MHz, CDCl₃): δ 9.16 (s, 1H), 8.60 (s, 1H), 8.51 (s, 1H), 8.13 (s, 1H), 7.79 (m, 1H), 7.68 (m, 1H), 7.45 (m, 1H), 7.35 (s, 1H), 7.33 (d, 1H, J=4 Hz), 7.25 (m, 1H), 7.13 (m, 1H), 7.01 (m, 1H), 6.98 (m, 1H), 6.19 (m, 1H), 5.31 (s, 2H), 4.35 (dd, 2H, J=8 Hz, J=16 Hz), 4.24 (m, 1H), 3.19 (m, 1H), 2.85 (m, 1H), 2.33 (m, 4H), 2.09 (m, 1H), 1.60 (t, 3H, J=8 Hz).

### Test Examples

### Biological Evaluation

### Example 1

### EGFR Cell Proliferation Inhibition Assay

The following in vitro assay was to determine the activity of the compounds of the present invention for inhibiting the proliferation of human epidermoid carcinoma A431 cells, which have high expression of EGFR.

The following in vitro assay was performed to determine the activity of the test compounds for inhibiting the proliferation of cancer cells, which have high expression of EGFR. The activity is represented by the IC₅₀ value. The general procedures of the assay were as follows: the cancer cells A431 highly expressing EGFR were chosen and seeded in a 96-well cell culture plate at a suitable density (e.g., 5000 cells/mL medium). The cells were then incubated in a carbon dioxide incubator until they reached 85% confluence. Then, the cell culture medium was replaced by fresh medium containing the test compounds with serial dilutions (general 6 to 7 concentrations). The cells were put back in the incubator and cultured continuously for 72 hours. The activity of the test compounds for inhibiting the cell proliferation was determined by using a Sulforhodamine B (SRB) method. IC₅₀ values were calculated by the rate of inhibition at various concentrations of the test compounds.

### The Activity of the Compounds of the Present Invention:

The biological activity of the compounds of the present invention was tested by using the assay described above. The IC₅₀ values were calculated and are shown in the table below:

| Example No. | IC₅₀ (EGFR/A431)(μM) |
|---|---|
| 1f | 0.045 |
| 2 | 0.031 |
| 3 | 0.029 |
| 7 | 0.038 |
| 8 | 0.015 |
| 9 | 0.049 |

Conclusion: Maleate and other salts and free base of the compounds of formula (I) have obvious activity in inhibiting the proliferation of A431 cells, which have high expression of EGFR.

### Example 2

### EGFR Kinase Activity Assay

The in vitro EGFR kinase activity was tested by the following assay.

The following assay was used to determine the activity of the compounds of the present invention for inhibiting EGFR kinase activity. The half maximal inhibitory concentration IC₅₀ (the concentration of the test compound showing 50% inhibition of the enzyme activity) of each compound was determined by incubating several different concentrations of the test compounds with a specific enzyme and substrate. EGFR kinase used in this assay was a human-derived recombinant protein, and was incubated with a peptide substrate at different concentrations of the test compounds in a buffer solution containing 60 mM HEPES (pH 7.5), 5 mM MgCl₂, 5 mM MnCl₂, 3 μM Na₃VO₄, 1.25 M DTT (1000×) and 20 μM ATP at 25° C., for 45 minutes. The EGFR kinase activity was determined quantitatively by using a time-resolved fluorescence method.

### The Activity of the Compounds of the Present Invention:

The biological activity of the compounds of the present invention was tested by using the assay described above. The IC₅₀ values were calculated and are shown in the table below:

| Example No. | IC₅₀ (EGFR/BIO) (μM) |
|---|---|
| 1f | 0.013 |
| 1 | 0.025 |
| 2 | 0.009 |
| 3 | 0.031 |
| 7 | 0.026 |
| 8 | 0.028 |
| 9 | 0.035 |

Conclusion: Maleate and other salts and free base of the compounds of formula (I) have obvious activity of inhibiting the EGFR Kinase activity.

### Example 3

### HER-2 Cell Activity Assay

The following in vitro assay was to determine the activity of the compounds of the present invention for inhibiting the proliferation of human carcinoma SK-BR-3 cells, which have high expression of HER-2.

The following in vitro assay was to determine the activity of the test compounds for inhibiting the angiogenesis and proliferation of cancer cells, which have high expression of HER-2. The activity is represented by the IC₅₀ value. The general procedure of the assay was as follows: the cancer cells line SK-BR-3 highly expressing HER-2 was chosen and the cells were seeded in a 96-well cell culture plate at a suitable density. The cells then were incubated in a carbon dioxide incubator until they reached 60% confluence. Then, the cell culture medium was replaced by fresh medium with the test compounds at various concentrations (6 to 7 concentrations). The cells were put back in the incubator and cultured continuously for 96 hours. The activity of the test compounds for inhibiting the cell proliferation was determined by using a Sulforhodamine B (SRB) method. IC₅₀ values of test cells were calculated by the rate of inhibition at various concentrations of the test compounds.

### The Activity of the Compounds of the Present Invention:

The biological activity of the compounds of the present invention was tested by using the assay described above. The IC₅₀ values were measured and are shown in the table below:

| Example No. | IC₅₀ (HER-2/SK-BR-3) (μM) |
|---|---|
| 1f | 0.059 |
| 1 | 0.064 |
| 2 | 0.049 |
| 3 | 0.056 |
| 7 | 0.045 |
| 8 | 0.078 |
| 9 | 0.069 |

Conclusion: Maleate and other salts and free base of the compounds of formula (I) have obvious activity in inhibiting the proliferation of SK-BR-3 cells highly expressing HER-2.

### Example 4

### HER-2 Kinase Activity Assay

The in vitro HER-2 kinase activity was tested by the following assay.

The following assay was used to determine the activity of the compounds of the present invention for inhibiting HER-2 kinase. The half maximal inhibitory concentration IC₅₀ (the concentration of the test compound showing 50% inhibition of the enzyme activity) of each compound was determined by incubating several different concentrations of the test compounds with a specific enzyme and substrate. HER-2 kinase used in this assay was a human-derived recombinant protein, and was incubated with a peptide substrate at different concentrations of the test compounds in a buffer solution containing 60 mM HEPES (pH 7.5), 5 mM MgCl₂, 5 mM MnCl₂, 3 μM Na₃VO₄, 1.25 M DTT (1000×) and 20 μM ATP at 25° C. for 45 minutes. The HER-2 kinase activity was determined by using a time-resolved fluorescence method.

### The Activity of the Compounds of the Present Invention:

The biological activity of the compounds of the present invention was tested by using the assay described above. The IC₅₀ values were calculated and are shown in the table below:

| Example No. | IC₅₀ (HER-2/SK-BR-3) (μM) |
|---|---|
| 1f | 0.065 |
| 1 | 0.053 |
| 2 | 0.028 |
| 3 | 0.071 |
| 7 | 0.064 |
| 8 | 0.053 |
| 9 | 0.046 |

Conclusion: Maleate and other salts and free base of the compounds of formula (I) have obvious activity for inhibiting the HER-2 Kinase activity.

### Solubility Assay

According to the conventional solubility measurement, the solubility of a compound of formula (I) and salts thereof was determined in three different systems: water, physiological saline and methanol. The results are shown in Table 1:

TABLE 1

| | Solubility Value (mg/mL) | | |
|---|---|---|---|
| Example | Physiological saline | 0.1M Hydrochloric acid | Water |
| Example 1f | 0.0026 | 1.43 | 0.008 |
| Example 1 | 0.19 | 23.33 | 12.49 |
| Example 2 | 0.36 | 54.88 | 1.52 |
| Example 3 | 0.08 | 12.65 | 17.25 |
| Example 7 | 0.10 | 31.93 | 5.39 |
| Example 8 | 3.32 | 32.4 | 22.41 |
| Example 9 | 0.2 | 16.59 | 0.15 |

Conclusion: Compared with free base and other salts of the compound of formula (I), the solubility of a maleate salt of the compound of formula (I) is significantly improved.

### Pharmacokinetics Assay

### Test Example 1

### Pharmacokinetics Assay of the Compounds of the Present Invention

### 1. Test Purpose

The rats were used as test animals. The compound of formula (I) and other salts thereof were administrated intragastrically, and a maleate salt of the compound of formula (I) was injected into the tail vein to determine the drug concentration in plasma at different time points by an LC/MS/MS method. The pharmacokinetic behavior, characteristics and the oral absolute bioavailability of the present compounds were studied and evaluated in rats.

### 2. Protocol

### 2.1 Test Samples

Example compound 1f, and Example compounds 1, 2, 4, 5, 6, 7 and 9.

### 2.2 Test Animals

28 healthy adult Sprague Dawley (SD) rats, half male and half female, were purchased from SINO-BRITSH SIPPR/BK LAB. ANIMAL LTD., CO, License number: SCXK (Shanghai) 2008-0016, and divided into 7 groups (4 rats in each group).

### 2.3 Equipments

TSQ Quantum Ultra AM triple quadrupole mass spectrometer, Thermo Finnigan (US); Agilent 1200 high performance liquid chromatography system, Agilent (US).

### 2.4 Preparation of the Test Compounds

The intravenous injection group: a suitable amount of compounds was weighed, dissolved in DMSO and diluted with normal saline to the final volume. The sample concentration was 2.5 mg/mL.

The intragastrical administration group: a suitable amount of compounds was weighed and added into 0.5% CMC-Na to prepare a 2.5 mg/mL suspension.

### 2.5 Administration

32 healthy adult SD rats, half male and half female, were divided into 7 groups (4 rats in each group). After an overnight fast, the example compound 1f, and example compound 1, 2, 4, 5, 6, 7 and 9 at a dose of 25 mg/kg (calculated by the base part) and a volume of 10 mL/kg were administered intragastrically or injected into the tail vein.

### 2.6 Sample Collection

For the intravenous injection group, blood samples (0.2 mL) were taken from orbital sinus before administration and at 2 min, 15 min, 30 min, 1.0 h, 2.0 h, 4.0 h, 6.0 h, 8.0 h, 12.0 h, 24.0 h and 36.0 h after administration, stored in heparinized tubes and centrifuged for 10 minutes at 3,500 rpm to separate blood plasma. The plasma samples were stored at −20° C.

For the intragastrical administration group, blood samples were taken before administration and at 0.5, 1.0, 2.0, 3.0, 4.0, 6.0, 8.0, 12.0, 24.0 and 36.0 hours after administration. The method to treat the samples was the same as with the intravenous injection group. The rats were fed 2 hours after administration.

### 3. Process

50 μL of rat plasma were added to 50 μL of a series of standard solutions respectively to obtain plasma concentration of 50.0, 100, 200, 500, 1000, 2000 and 5000 ng/mL, then the plasmas at various concentrations were mixed with 50 μL of methanol for 3 min by using a vortexer, and the mixture was centrifuged for 10 minutes (13500 rpm/min). 10 μL of the supernatant were analyzed by LC-MS/MS.

### 2.9 Calculation of the Pharmacokinetic Parameters

### 4. Results of Pharmacokinetic Parameters

Pharmacokinetic parameters of the compounds of the present invention were shown in Table 2.

TABLE 2

| | | Cₘₐₓ | AUC₀₋ₜ | t_{1/2} | MRT | CL/F | Vz/F |
|---|---|---|---|---|---|---|---|
| Compound | F (%) | (μg/mL) | (μg · h/mL) | (h) | (h) | (mL/min/kg) | (1/kg) |
| Example 1f | 42.2 | Gavage | 52.81 ± 23.59 | 3.4 ± 0.45 | 7.66 ± 0.75 | 0.53 ± 0.18 | 2.6 ± 0.94 |
| | | 6.47 ± 1.81 | | | | | |
| | | Vein | 125.4 ± 38.95 | 5.25 ± 2.01 | 4.22 ± 0.71 | 0.21 ± 0.07 | 1.72 ± 1.1 |
| Example 1 | 47.1 | Gavage | 104.58 ± 56.54 | 4.4 ± 1.3 | 8.93 ± 1.39 | 0.31 ± 0.16 | 1.91 ± 1.24 |
| | | 8.99 ± 4.87 | | | | | |
| | | Vein | 221.86 ± 76.99 | 4.92 ± 0.6 | 5.5 ± 0.99 | 0.12 ± 0.043 | 0.89 ± 0.39 |
| Example 2 | 48.3 | Gavage | 116.48 ± 67.23 | 4.45 ± 1.63 | 8.03 ± 0.99 | 0.29 ± 0.19 | 1.84 ± 1.19 |
| | | 10.19 ± 4.41 | | | | | |
| | | Vein | 241.16 ± 92.12 | 3.74 ± 1.20 | 2.96 ± 0.47 | 0.48 ± 0.2 | 2.84 ± 1.88 |
| Example 4 | 63.2 | Gavage | 92.27 ± 36.97 | 3.67 ± 0.83 | 8.4 ± 0.65 | 0.32 ± 0.16 | 1.6 ± 0.54 |
| | | 6.92 ± 1.82 | | | | | |
| | | Vein | 145.75 ± 40.35 | 3.77 ± 0.22 | 5.46 ± 0.48 | 0.18 ± 0.04 | 0.99 ± 0.26 |
| Example 5 | 72.1 | Gavage | 81.53 ± 40.46 | 3.46 ± 0.56 | 7.95 ± 0.61 | 0.38 ± 0.19 | 1.81 ± 0.84 |
| | | 7.88 ± 2.51 | | | | | |
| | | Vein | 112.84 ± 62.1 | 3.12 ± 0.37 | 4.2 ± 0.99 | 0.3 ± 0.22 | 1.33 ± 0.88 |
| Example 6 | 50.5 | Gavage | 81.72 ± 22.16 | 4.15 ± 0.09 | 8.59 ± 0.22 | 0.32 ± 0.09 | 1.95 ± 0.57 |
| | | 6.27 ± 1.58 | | | | | |
| | | Vein | 162.28 ± 55.65 | 5.27 ± 0.29 | 4.94 ± 0.58 | 0.17 ± 0.04 | 1.25 ± 0.31 |
| Example 7 | 39.2 | Gavage | 44.34 ± 16.01 | 3.52 ± 0.69 | 7.9 ± 0.74 | 0.63 ± 0.27 | 3.23 ± 1.65 |
| | | 5.16 ± 1.64 | | | | | |
| | | Vein | 112.84 ± 62.1 | 3.12 ± 0.37 | 4.2 ± 0.99 | 0.3 ± 0.22 | 1.33 ± 0.88 |
| Example 9 | 57.6 | Gavage | 66.12 ± 17.17 | 3.47 ± 0.18 | 7.96 ± 0.7 | 0.4 ± 0.12 | 1.99 ± 0.49 |
| | | 5.87 ± 1.61 | | | | | |
| | | Vein | 115.15 ± 33.06 | 4.75 ± 0.32 | 5.54 ± 0.58 | 0.23 ± 0.06 | 1.58 ± 0.46 |

### CONCLUSION

Compared with free base and other salts of the compound of formula (I), the maleate salt of the compound of formula (I) has significant improvement in pharmacokinetic characteristics and bioavailability, and has an obvious pharmacokinetic advantage.

1. A pharmaceutically acceptable salt of (E)-N-[4-[[3-chloro-4-(2-pyridylmethoxy) phenyl]amino]-3-cyano-7-ethoxy-6-quinolyl]-3-[(2R)-1-methylpyrrolidin-2-yl]prop-2-enamide of formula (I): 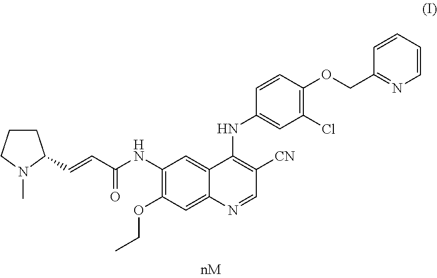
wherein:
n is 1, 2 or 3; and
M is an acid molecule.

2. The salt according to claim 1, wherein said salt is an inorganic salt.

3. The salt according to claim 2, wherein said inorganic salt is selected from the group consisting of phosphate, hydrochloride salt, sulfate, nitrate and hydrobromide salt.

4. The salt according to claim 3, wherein said inorganic salt is the hydrochloride salt.

5. The salt according to claim 4, wherein n is 2.

6. The salt according to claim 1, wherein said salt is an organic salt.

7. The salt according to claim 6, wherein said organic salt is selected from the group consisting of p-toluenesulfonate, methanesulfonate, maleate, tartrate, succinate, acetate, trifluoroacetate, fumarate, citrate, benzenesulfonate, benzoate, naphthalenesulfonate, lactate and L-malate.

8. The salt according to claim 7, wherein said salt is maleate.

9. The salt according to claim 8, wherein n is 2.

10. A process of preparing a pharmaceutically acceptable salt according to claim 1, comprising a step of reacting (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide with a corresponding acid to form the salt.

11. The process according to claim 10, wherein said acid is an inorganic acid or an organic acid selected from the group consisting of phosphoric acid, hydrochloric acid, sulfuric acid, nitric acid, hydrobromic acid, p-toluenesulfonic acid, methanesulfonic acid, maleic acid, tartaric acid, succinic acid, acetic acid, trifluoroacetic acid, fumaric acid, citric acid, benzenesulfonic acid, benzoic acid, naphthalenesulfonic acid, lactic acid and L-malic acid.

12. A pharmaceutical composition comprising a therapeutically effective amount of the pharmaceutically acceptable salt according to claim 1 and a pharmaceutically acceptable carrier.

13. A process of preparing the pharmaceutical composition according to claim 12, comprising a step of combining the pharmaceutically acceptable salt according to claim 1 with the pharmaceutically acceptable carrier or a diluent.

14-15. (canceled)

16. A method of treating a protein kinase related disease in a subject in need thereof, comprising administering to the subject the pharmaceutical composition according to claim 12, wherein the protein kinase is selected from the group consisting of EGFR receptor tyrosine kinases and HER-2 receptor tyrosine kinases.

17. A method of treating a cancer in a subject in need thereof, comprising administering to the subject the pharmaceutical composition according to claim 12, wherein the cancer is selected from the group consisting of lung cancer, breast cancer, squamous cell carcinoma and stomach cancer.

18. The pharmaceutical composition according to claim 12, wherein in the pharmaceutically acceptable salt, n is 2.

19. The pharmaceutical composition according to claim 18, wherein in the pharmaceutically acceptable salt, the salt is hydrochloride salt or maleate.

20. The method according to claim 13, wherein in the pharmaceutically acceptable salt, n is 2.

21. The method according to claim 20, wherein in the pharmaceutically acceptable salt, the salt is hydrochloride salt or maleate.

22. The method according to claim 17, wherein in the pharmaceutically acceptable salt, n is 2.

23. The method according to claim 22, wherein in the pharmaceutically acceptable salt, the salt is hydrochloride salt or maleate.
